(19) United States
(12) Patent Application Publication
(10) Pub. No.: US 2015/0272596 A1
(43) Pub. Date: Oct. 1, 2015

(54) MRI COMPATIBLE SURGICAL MOTOR-POWERED DRIVERS AND RELATED METHODS

(71) Applicant: MRl lnterventions, Irvine, CA (US)

(72) Inventors: Kamal Vij, Chandler, AZ (US); Rajesh Pandey, Irvine, CA (US); Jesse Flores, Perris, CA (US)

(21) Appl. No.: 14/638,273

(22) Filed: Mar. 4, 2015

Related U.S. Application Data
(60) Provisional application No. 61/971,139, filed on Mar. 27, 2014.

(51) Int. Cl. A61B 17/17; G01R 33/28; A61B 5/055; A61B 17/16; A61B 17/88

(52) U.S. Cl. CPC A61B 17/1703; A61B 17/162; A61B 17/1628; A61B 17/1615; A61B 17/8875; A61B 17/1626; A61B 17/1622; A61B 5/0555; G01R 33/285; A61B 2017/00911

(57) ABSTRACT

A surgical, motor-powered hand-held MRI-compatible drill with a remote control unit and user controls for drilling though target bone of a patient and/or attaching a bone screw.

### RELATED APPLICATIONS

This application claims the benefit of and priority to U.S. Provisional Application Ser. No. 61/971,139, filed Mar. 27, 2014, the contents of which are hereby incorporated by reference as if recited in full herein.

### FIELD OF THE INVENTION

The present invention relates to medical devices and, more particularly, tools and methods for drilling in or through bone of a patient and/or driving screws.

### BACKGROUND OF THE INVENTION

During MRI-guided surgeries, it can be desired to drill through bone such as a skull to define a surgical path for passing medical interventional devices and/or to insert screws into bone.

### SUMMARY OF THE INVENTION

Embodiments of the present invention are directed to surgical, motor-powered drivers (which can be for drilling into bone and/or driving screws into bone) that can be safely used in an MRI environment, including proximate the high-field magnet while a patient is on a bed/gantry of an MR Scanner.

Embodiments of the invention are directed to surgical, motor-powered drivers. The drivers include: a driver handpiece comprising a non-ferromagnetic housing, wherein the driver housing is sterile for surgical use; a non-magnetic motor in the housing; a shaft in communication with the motor; and a chuck extending from the housing adapted to serially, releasably hold a drill bit and a screw driver so that the drill bit or screw driver extends out from the chuck and is rotated by the motor.

The driver can include a control unit connected to the handpiece by a cable having a length of at least five feet to allow the control unit to be positioned remotely from the handpiece outside a gauss line of an MRI suite while the handpiece is held in a magnetic field of a magnet of an MRI scanner.

The handpiece can operate the shaft with between about 20-150 rpm and can generate a maximum torque of about 6.2 in-lb.

The driver can include a speed increase gear train in the handpiece in communication with the motor, wherein the gear train has a gear ratio of 2:1 and can generate an output speed between about 40-300 rpm with a maximum torque of about 3.1 in-lb.

The handpiece can have a barrel attached to a position-adjustable handle, wherein the handle can rotate from lockable orientations between about 0-90 degrees to be respectively in-line with the barrel or substantially orthogonal with the barrel.

The driver can have a dual operating mode, including a drill mode and a screw driver mode. The screw driver mode can have a lower speed than the drill mode.

The driver can include a user interface control on the driver handpiece configured to allow a user to switch between drill and screw driver modes.

The driver can include at least one footswitch with a respective pedal attached to the control unit with a cable.

The handpiece can include a trigger to direct the driver to operate.

The handpiece can be reusable in sterile medical environments and can be configured to withstand a plurality of autoclaving and/or Ethylene Oxide (“EtO”) sterilization processes so as to remain functional and not deteriorate.

The footswitch can have a non-ferromagnetic enclosure with cooperating housing members that can move relative to each other to allow the pedal to operate. A gasket and/or O-ring can reside between the cooperating members allowing the relative movement to inhibit liquid entry into the enclosure to thereby provide a splash-resistant configuration.

The driver can include a cable attached to the handpiece to connect the motor to a control unit. The cable and handpiece can be configured to be reusable and withstand a plurality of sterilizations.

The handpiece can have a barrel and a handle. The handle can be pivotably attached to the barrel at a pivot on a rear end portion of the barrel. The handle can have a channel that slidably travels over a curved outer surface of the barrel a distance away from the pivot.

The curved outer surface can include a fin that projects radially outward a distance beyond an adjacent curved surface of the rear end portion of the barrel.

The driver can include an indexing control mechanism configured to allow a user to controllably adjust an orientation of the handle relative to the barrel over a plurality of positions.

The fin can have a circumferentially extending slot that receives a pin of the indexing control mechanism.

The driver can include a control unit remote from the handpiece and a connector cable attached to and connecting the handpiece and the control unit. The control unit can have a controller that controls operation of the motor including forward/reverse operation, speed control, start/stop and system power.

The control unit can have a housing of non-ferromagnetic material with connections for main power and the connector cable.

Other embodiments are directed to methods of inserting a bone screw or forming an aperture in bone of a patient during an MRI guided surgical procedure. The methods include: (a) placing a patient on a patient support surface in an MR Scanner room; (b) holding a motor-driven driver against a target location of a patient while the patient is in the MR Scanner room; (c) electrically powering the driver; and (d) drilling an aperture in target bone of the patient or driving a bone screw into bone of a patient in response to the electrically powering step.

The method can include allowing a user to depress at least one foot switch in communication with the driver to cause the driver to carry-out step (d).

The motor can be a non-magnetic motor that can operate at between about 20-150 rpm with a maximum torque of about 6.2 in-lb.

The driver can have a speed increase gear train with a gear ratio of 2:1 that can generate a speed between about 40-300 rpm with a maximum torque of about 3.1 in-lb.

The driver can have a “pistol shape” with a barrel and a rotatably adjustable handle orientation, that can be locked into different orientations, typically rotatable between about 0-90 degrees to be substantially orthogonal with the barrel to substantially in-line with the barrel.

The powered driver can have a dual operating mode, including a drill mode (higher speed) and a driver mode (lower speed that the drill mode). A user interface on the driver can allow a user to switch between modes.

The powered driver can optionally be operated using one or more foot pedals attached to the powered driver via cabling.

The powered driver can optionally be operated in other manners such as using a trigger on the power driver itself or via a remote hand trigger.

The powered driver can be attached to a driver control unit with a controller for directing operation of the motor. The control unit may be positioned outside a defined gauss limit line in the Scanner room holding the high-field magnet.

Bevel and pinion gears of the speed reduction gear train, where used, can comprise acetal material.

The housing, shaft, and chuck can comprise Polyetheretherketone (PEEK) components.

The power driver can be reusable in a sterile medical environment and configured to withstand a plurality of autoclaving and/or EtO sterilization processes so as to remain functional and not deteriorate.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1 is a partial assembly view of a powered surgical drill system according to embodiments of the present invention.

FIGS. 2A and 2B are side views of an exemplary power drill with a handle that can be moved to different orientations according to embodiments of the present invention.

FIG. 3A is a greatly enlarged side perspective view of a powered surgical drill according to embodiments of the present invention.

FIG. 3B is a partial section view of the drill shown in FIG. 3A according to embodiments of the present invention.

FIG. 3C is a side view of the device shown in FIG. 3B.

FIG. 4 is an enlarged top view of an exemplary control unit according to embodiments of the present invention.

FIG. 5A is a top view of an exemplary footswitch configuration with associated cable according to embodiments of the present invention.

FIG. 5B is a top view with one of the tops of the footswitches shown off the bottom according to embodiments of the present invention.

FIGS. 5C and 5D are side perspective views of the device shown in FIG. 5B according to embodiments of the present invention.

FIG. 5E is an end view of the foot switch shown in FIG. 5B illustrating the wire entry portal according to embodiments of the present invention.

FIG. 6 is a top view of an exemplary control cable that can connect the drive hand piece to a control unit according to embodiments of the present invention.

FIG. 7A is an end view of a powered driver used on a patient while positioned proximate a magnet of an MRI scanner according to embodiments of the present invention.

FIG. 7B is a side view of a powered driver used on a patient while positioned proximate a magnet of an MRI scanner according to embodiments of the present invention.

FIGS. 8 and 9 are side perspective views of a powered driver used to insert a bone screw into skull bone of a patient to secure a base of a trajectory frame thereto according to embodiments of the present invention.

### DETAILED DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art. The term “Fig.” (whether in all capital letters or not) is used interchangeably with the word “Figure” as an abbreviation thereof in the specification and drawings.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

The term “about” refers to numbers in a range of +/−20% of the noted value.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

The term “light-weight” refers to power drivers that weigh less than about 2 pounds. The term “MRI compatible” means that the so-called component(s) are safe for use in an MRI environment (e.g., in a high magnetic field of an MRI scanner) and are typically made of non-ferromagnetic MRI compatible material(s) suitable to reside and/or operate in a higher magnetic field environment. The term “high magnetic field” refers to field strengths above about 0.5T, typically between 1.5T and 10T, e.g., typically 1.5T, 2T, 3T, associated with MRI/MR Scanners. The term “gantry” refers to a device holding imaging components about a patient portal. The gantry can be relatively short for CT Scanners and may be part of a cylindrical patient space for some MRI Scanners. The gantry may hold or reside about rails of a patient support of an MRI Scanner and may include the patient table or other structure.

The terms “MRI Scanner” or “MR Scanner” are used interchangeably to refer to a Magnetic Resonance Imaging system and includes the magnet, the operating components, e.g., RF amplifier, gradient amplifiers and operational circuitry including, for example, processors (the latter of which may be held in a control cabinet) that direct the pulse sequences, select the scan planes and obtain MR data.

The term “RF safe” means that the device is configured to operate safely when exposed to RF signals, particularly RF signals associated with MRI systems, without inducing unplanned current that inadvertently unduly heats local tissue or interferes with the planned therapy.

The term “sterile” means that the device meets or exceeds surgical cleanliness standards.

The term “chuck” refers to a type of clamp used to releasably hold a rotating tool (such as screw driver or drill bit). The chuck may be of any suitable type including, for example, a Jacobs style chuck as shown in the figures or other chuck configurations. The chuck may have jaws arranged in a radially symmetrical pattern to hold the tool.

According to embodiments of the present invention, surgical drills and methods for using the same are provided for forming a surgical entry path into or through bone of a patient and/or attaching bone screws. According to some embodiments, the drills and methods are used or usable to form an access path through a patient's skull and/or attaching bone screws thereto.

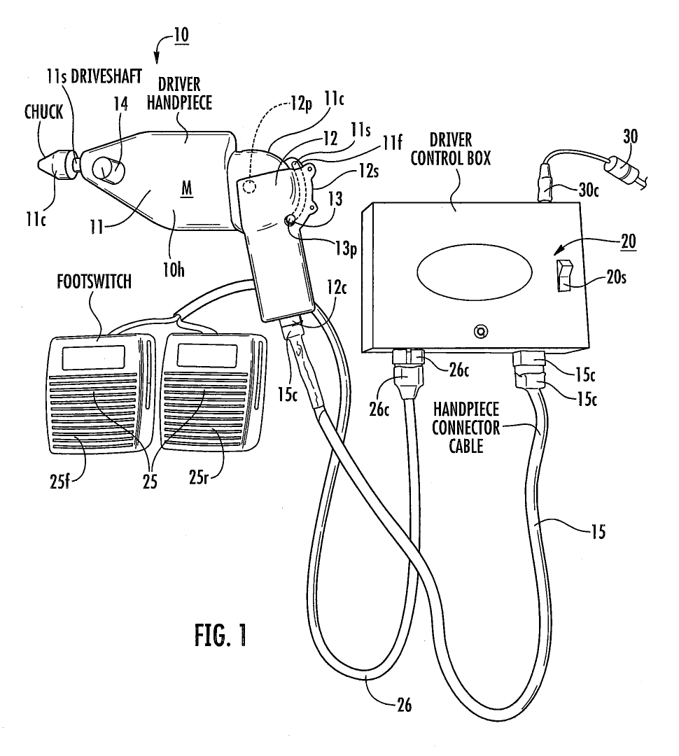

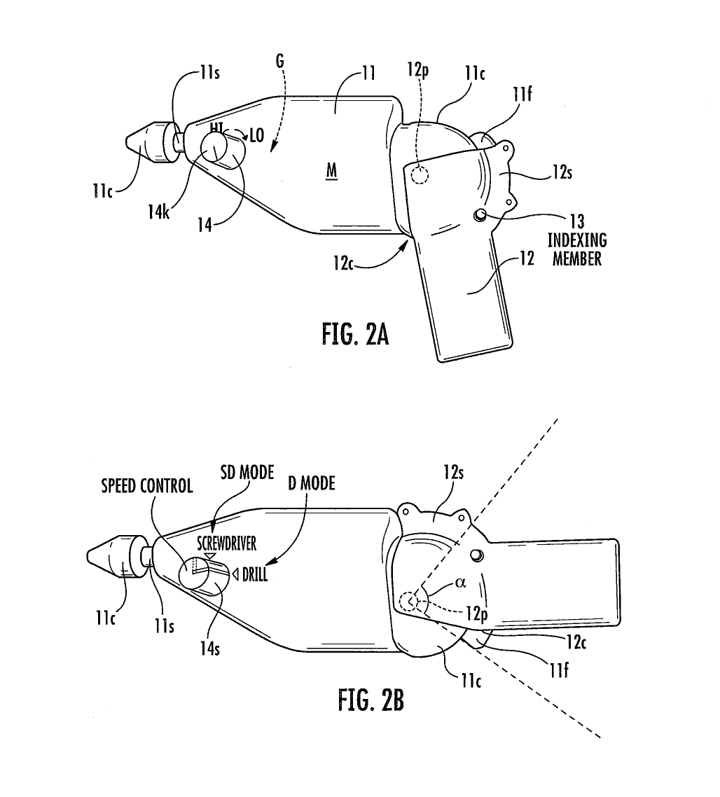

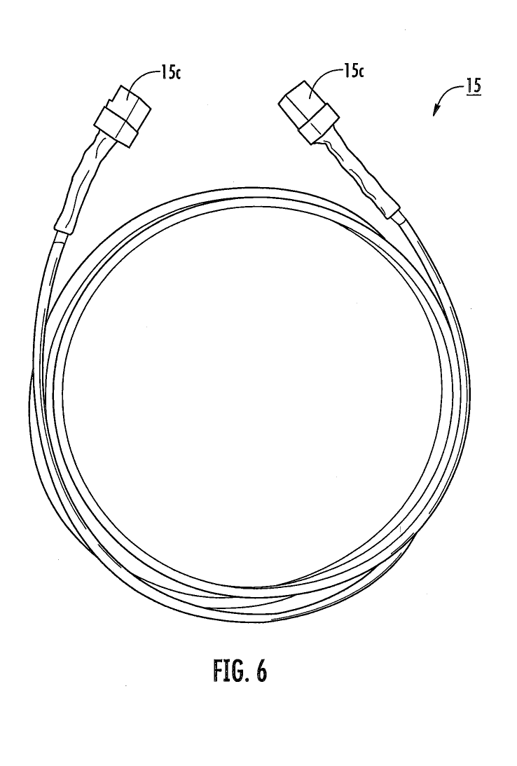

FIGS. 1-6 illustrate embodiments of a power driver 10 and related components. As shown, the power driver 10 includes a housing 10h with a driver body (e.g., “barrel”) 11 with a handle 12 that can optionally pivot into different lockable orientations. For example, the handle 12 can angle downward from the driver body 11 into a “pistol” type grip orientation as shown in FIG. 2A and/or can be selectively positioned to have an orientation that is in line with the driver body 11 as shown in FIG. 2B.

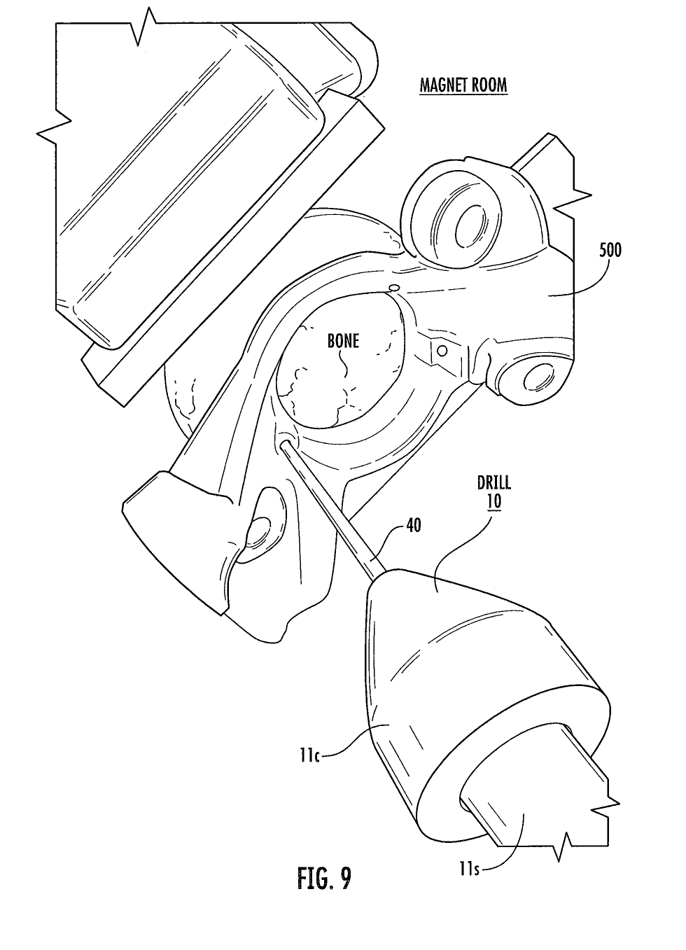

The power driver 10 can include a drive shaft 11s that connects to a chuck 11c for releasably holding drill bits or screw drivers 40 (FIGS. 8, 9). The power driver 10 can include a cord (also interchangeably called a “cable”) 15 with connectors 15c. The power driver 10 can include an on-board motor M. One end of the cord 15 can connect via connector 15c to a connector 12c on the driver, typically on the handle 12, such as, for example, at a lower end of the handle 12. The other end of the cord 15 can have a connector 15c that connects to a driver control unit 20. The cord 15 can releasably attach to both the driver 10 and the driver control unit 20.

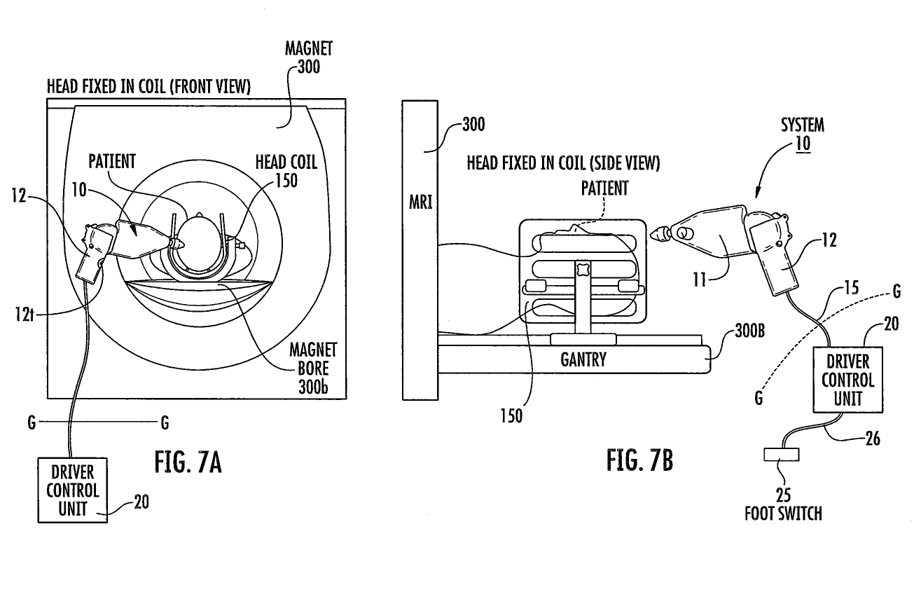

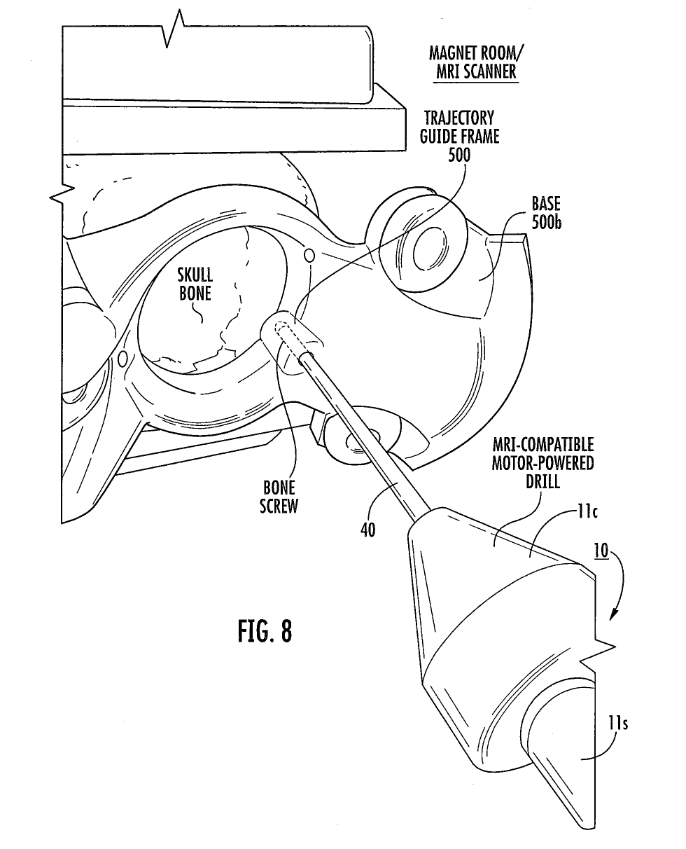

The power driver 10 can include at least one user control to allow for operational adjustments, such as speed and/or forward and reverse drill and/or screw directions. The user control can include a speed control input 14 that may reside on the driver body 11, as shown. However, it may also be incorporated into a trigger 12t (FIG. 7A) or be provided at a different location including on or in the drive control unit 20. The power driver hand piece 10 (barrel 11 and handle 12) can be light weight, typically between 0.5 pounds and 2 pounds. In some embodiments, the driver hand piece 10 weighs about 1.5 pounds. The driver hand piece 10 can have a primary body that encloses a motor and a gear train therein.

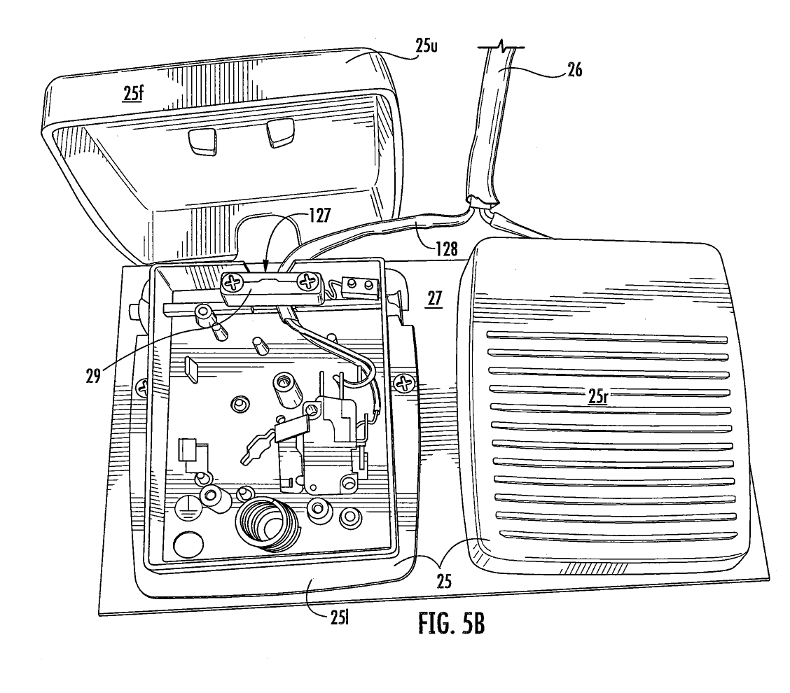

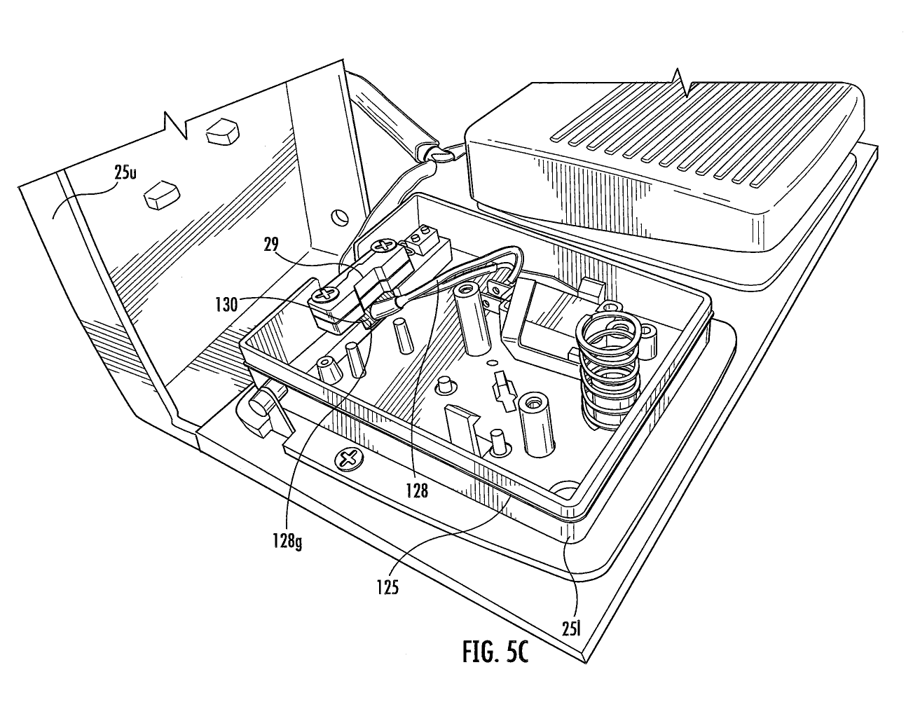

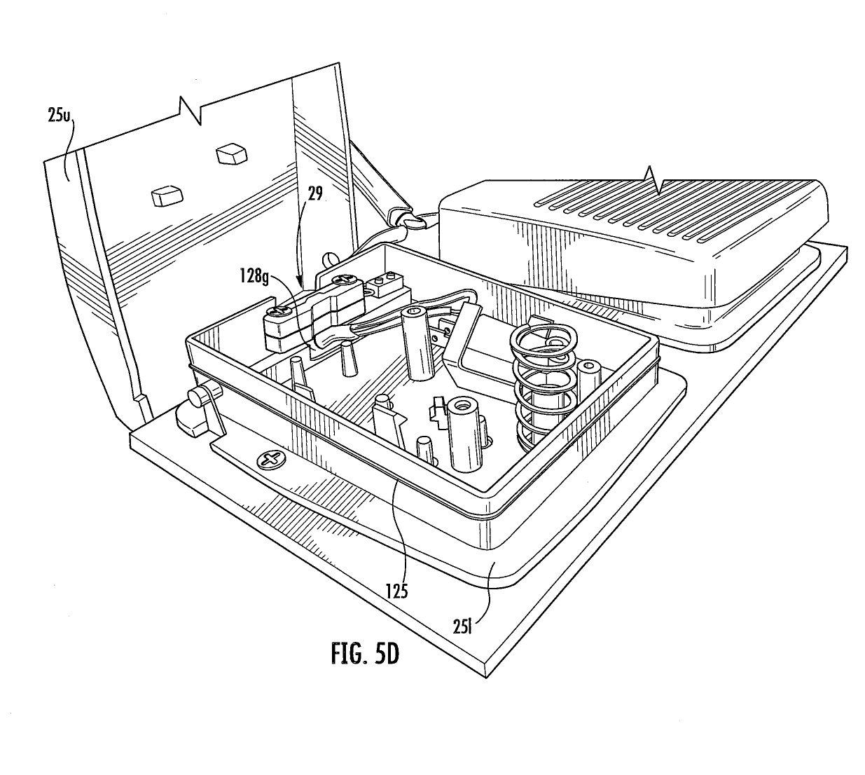

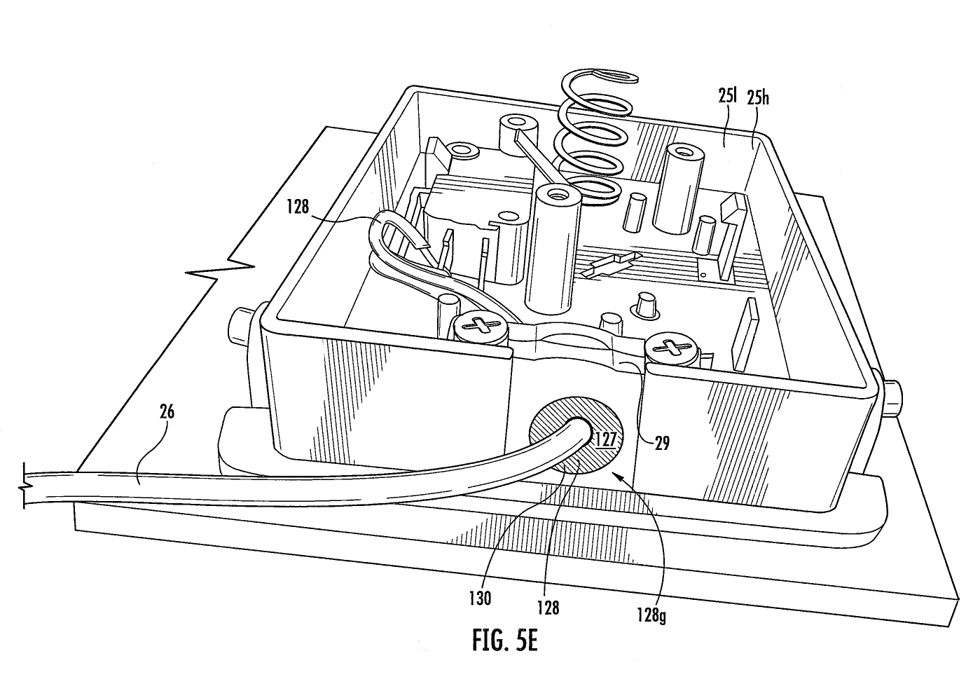

The user control can comprise at least one foot pedal 25 as shown in FIG. 1 or a trigger 12t, typically on the handle of the driver 10 (FIG. 7A). In the embodiment shown in FIG. 1, the user control includes two foot switches: one that controls a forward direction, and the other a reverse direction of the driveshaft 11s. However, a single foot switch may provide both actions. The foot switch(es) 25 can also attach to the driver control unit 20 typically via cord 26. Connectors 26c can allow the cord 26 to engage the driver control unit 20. The connectors 26c may be configured to releasably attach to the driver control unit 20. Where used, the footswitch or foot switches 25 can be held on a planar mounting plate 27 (FIG. 5A).

Where the handle 12 is provided as a moveable component, as shown, the handle 12 can be pivotably attached to the driver body 11 via pivot 12p. A lockable indexing mechanism 13 that is attached to both the driver body 11 and the handle 12 can be used to selectively control the handle 12 movement so as to be able to rotate and engage the driver body 11 at desired orientations. The indexing mechanism 13 can be a push-button feature that allows the user to rotate the driver handle 12 from between about 0 to 90 degrees, with lockable orientations in increments in between. As shown in FIG. 1, the driver barrel 11 can include a circumferentially extending slot 11s that cooperates with at least one pin 13p to provide the indexing mechanism.

The indexing mechanism 13 can be configured as cooperating first and second titanium pins and a 300-series SST spring that cooperate with a segment of the barrel 11. However, other indexing mechanism configurations may be used, where included in the device 10.

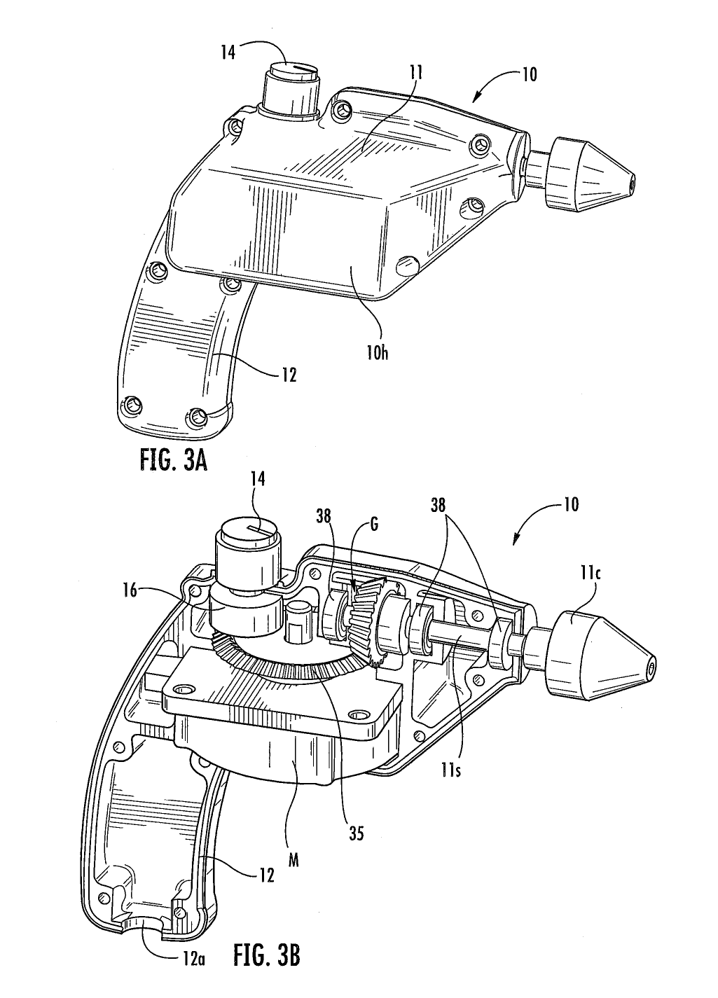

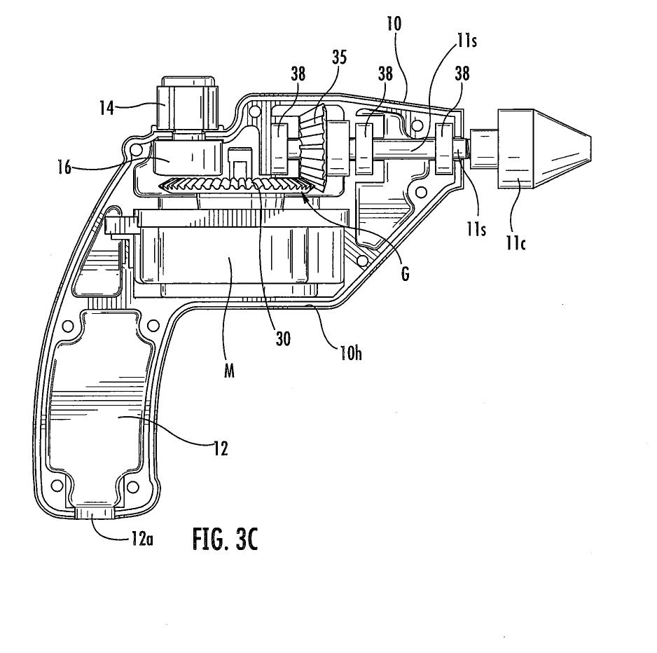

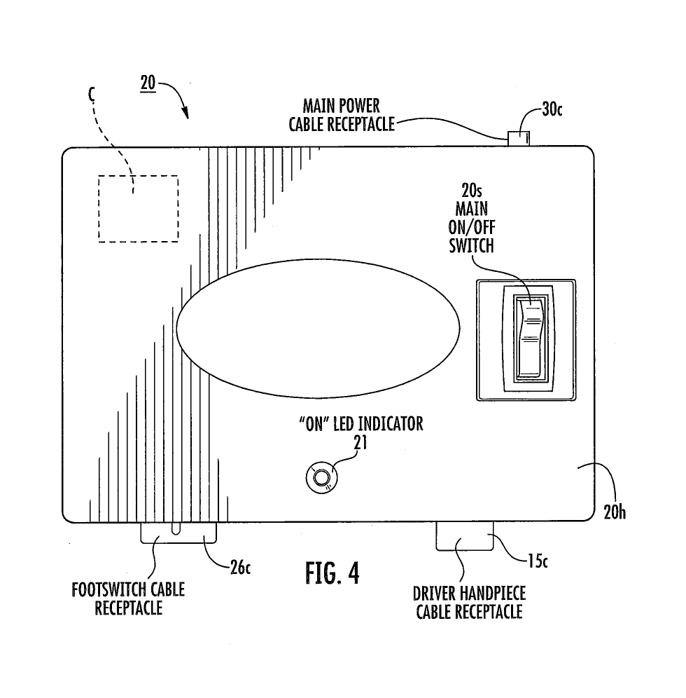

The handle 12 can have a shoulder that slidably rotates over a curved surface 11c at a rear end of the driver body. The curved surface 11e can merge into an outwardly projecting fin 11f. The fin 11f can be snugly received in the shoulder 12s. The handle 12 can include a channel 12c that slidably receives the fin 11f. The fin 11f can extend an angular distance a (FIG. 3) that is between about 30-120 degrees, typically about 45 degrees to about 90 degrees.

The driver 10 (e.g., driver body 11 and/or handpiece 12) can contain a non-magnetic motor M (such as for example a motor sold by Shinsei Motor Corporation, Model S3N-USR60), a drive train, electrical wiring, an optional trigger 12t (FIG. 7A), speed control knob or switch 14, and the connector receptacle 12c.

A motor should be selected that has high enough speed to drill efficiently, but also high enough torque to drive screws effectively. The torque at which screws reach full tightness in bone (tight enough to withstand at least 25 lb of pull force) is about 2.4 in-lb. A very efficient drilling speed for bone is between about 250 rpm to about 300 rpm. In some embodiments, a minimum torque for drilling is about 1.5 in-lb. It is preferred that the driver 10 does not have enough torque (e.g., is configured to generate a max torque of about 3 in-lb) to break or strip the screw heads when driving—effectively creating a “self-limiting” screw driver.

The motor M can have an operational speed range of between about 20-150 rpm, with a maximum torque of about 6.2 in-lb. The speed range is suitable for screw driving but is typically too slow for drilling. The maximum torque can be selected to be sufficiently high to strip the screw heads, and even to break most screw designs. However, in some embodiments, the driver 10 can include a 2:1 speed-increase gear train G (FIG. 2A, 3B, 3C) in communication with the motor M (closer to the shaft 11s) to bring the maximum speed up to the desired drilling speed, and to reduce the torque to avoid breaking screws. The torque is high enough to insert screws and to drill effectively. In some embodiments, the driver 10 can have a speed with the 2:1 gearing that is between about 40 rpm to about 300 rpm and the maximum torque can be about 3.1 in-lb.

The drive shaft 11s and chuck 11e can be made from PEEK. The drive shaft 11s can be the motor shaft or a separate shaft attached to the motor/motor shaft and does not require a gear train. Where used, the gears/gear train G can be made from any suitably hard non-ferromagnetic material, e.g., one or more of glass-filled acetal, brass, aluminum, or 316L SST. The housing 10h can be made of plastic or a polymeric material (e.g., PEEK, Ultem, or Nylon). The housing 10h may also or alternatively be made of anodized aluminum. Fasteners for the housing 10h can be made from brass, 316L SST, aluminum, or titanium. Any of these materials can make the driver 10 MRI safe.

As shown in FIGS. 2A and 2B, the driver 10 can have a speed control member 14 that can be adjusted by a knob 14k or switch, for example, that allows continuous variable speed (FIG. 2A), or by a switch 14s (FIG. 2B) that moves between a “Drill mode”, shown as a D mode (high speed) and a “Screw Driver mode” (lower speed), shown as a SD mode. The dual-speed switch may provide more user control as driving screws at a speed above 150 rpm can cause stripping of bone. The speed control member 14, e.g., switch 14s, can set the speed in “Screw Driver mode” at between about 100 rpm to about 140 rpm. For “Drill mode”, the switch 14s can set the speed higher, typically between about 250 rpm to about 300 rpm.

The driver 10 is in the sterile field, so it is preferably configured to withstand repeated sterilizations for multiple uses. Thus, the materials for the housing 10h, wires, switch 14, and connector receptacle 12c should withstand 270° F. and/or EtO for a suitable time period for medical sterility standards, multiple times. Ethylene Oxide (EtO) sterilization is mainly used to sterilize medical and pharmaceutical products that cannot support conventional high temperature steam. Also, the driver function is configured so as to functionally operate and not deteriorate with exposure to moisture from an autoclave and/or EtO sterilization cycle. So anything that is moisture-sensitive in or on the device 10 can be encapsulated in potting material.

FIGS. 3A-3C illustrate that the speed control member 14 can be oriented to be upright at a top of the housing 10h above the handle 12. FIGS. 3B and 3C illustrate that the motor M can reside under the gear train G. The gear train G can include a bevel gear 30 and a cooperating pinion gear 35. The shaft 11s can hold a plurality of axially spaced apart bearings 38. As shown, one bearing 38 can reside on an internal (innermost) end portion of the shaft 11s and two bearings 38 can reside axially spaced apart with the pinion gear 35 between the internal innermost bearing and the other two bearings 38. One bearing 38 can reside in the housing 10h adjacent the chuck 11e and the other can reside closer to the pinion gear 35 to provide for load balance according to some embodiments.

FIGS. 3B and 3C also illustrate that a potentiometer 16 can reside under the speed control member 14, e.g., switch 14s, above the bevel gear 30 and motor M.

FIGS. 3B and 3C further illustrate that the lower end portion of the handle 12 can include a channel or aperture 12a for a cord or connector (15, 15c, FIG. 1) that can electrically connect the powered hand-held driver to the motor and speed controller or driver control box 20 (FIG. 1).

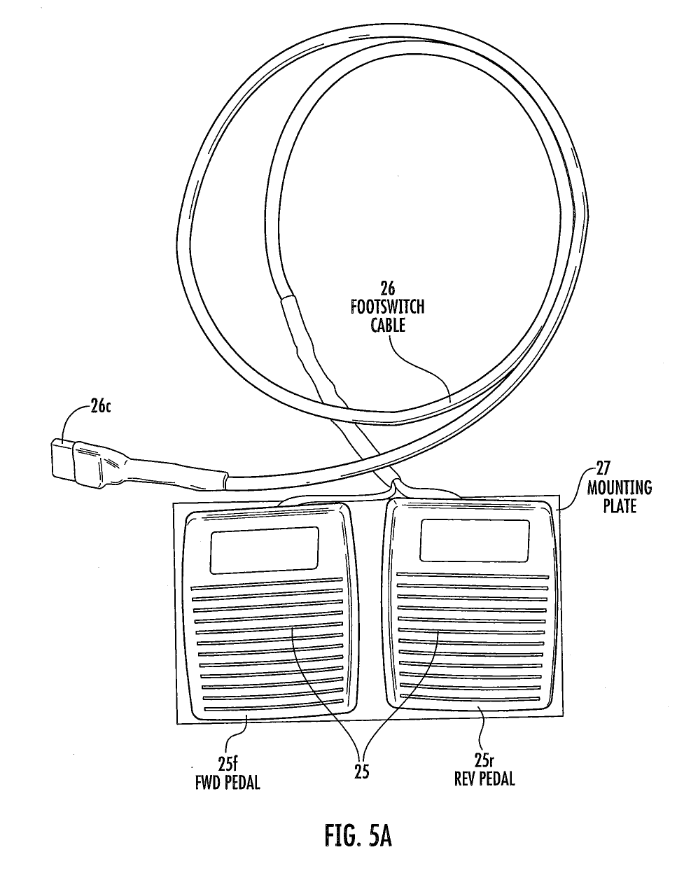

Referring to FIGS. 1 and 4, the driver control unit 20 can contain part of all of the controller C (such as a controller from Shinsei Motor Corporation, Model 6060) for the handpiece/driver motor M. The controller C regulates operation of the motor M. It can include connections for motor commutation, forward/reverse operation, speed control, start/stop, and system power. The controller C can be enclosed in a housing 20h that can be plastic and/or elastomeric that has receptacles for mains power 30c, the connector cable 15c, and the footswitch 26c, where used. The control unit 20 is not required to have any external hard-wired cables to allow it to be easy to pick up and move around the Scanner room (and in and out of the Scanner room) as needed.

Also, the length of different cables can be adjusted easily depending on users' needs for their particular scanner room. The control unit 20 typically remains away from the Scanner, beyond the gauss line. The control unit 20 can have a main on/off power switch 20s and an indicator light 21 that lights up when mains power is ON.

The footswitch 25, where used, can comprise two pedals: one for forward operation 25f and one for reverse operation 25r of the motor M. The footswitch(es) 25 can be connected to the control unit 20 via a cable 25 that plugs into a receptacle 26c on the control unit 20. The cable 26 can be removably attached to the control unit 20. The cable 25 is typically hardwired to the footswitch(es) 25. The cable 25 can have a length that is about five feet, but can be longer to allow the control unit 20 to be positioned further away from the scanner magnet 300 (FIGS. 7A, 7B). The foot switch(es) are typically pedals of on/off switches, so that when force is applied to the pedal by a user/foot, the motor M turns at a set speed. The foot pedals can be mounted to a mounting plate 27 (FIG. 5A, 5D) which can be non-ferromagnetic, e.g., an aluminum (MRI safe) plate. The pedals 25 can have external polymeric and/or plastic enclosures, and the fasteners (e.g., springs, screws) and switch plates can be made from non-ferromagnetic material, such as, for example, from beryllium copper, 316L SST, brass or 316L SST, The pedals may also be enclosed in anodized aluminum or 316L SST. Any of these materials makes the footswitch(es) 25 MRI Safe. The footswitch 25 can be configured to accommodate cleaning with liquid disinfectants and is splash-resistant.

As shown in FIGS. 5B-5D, for example, to make the footswitch 25 splash-resistant, each can have a seal member, such as at least one gasket or O-ring 125 positioned about a perimeter of the upper or lower housing member 25u, 25l, respectively, typically residing on the outer wall of the lower housing member 25l. However, an O-ring, gasket or other seal member 125 may also or alternatively reside against a perimeter of the inner wall of the upper housing member 25u. Thus, the seal member 125 can reside between the moving parts of the enclosure of the footswitch 25. The seal 125 may be a silicone O-ring. The seal 125 is not required to totally or hermetically seal the housing of a respective footswitch 25 but can provide a liquid or splash-resistant configuration for the interior components such as internal wires 128 and components, e.g., electrical connections to the drive(s) from the cable 26.

FIGS. 5C-5E illustrate the wire pass through or portal 127 can be a filled or closed portal. The wires 128 that extend from/to the connector cable 26 can be passed through a gasket or other sleeve and inserted into a clamp block or portal 127. Prior to clamping, the gasket or sleeve 128g can be filled with silicone RTV or other filler 130 to fill voids between the inner diameter of the (clamped) gasket or sleeve 128g and the wires 128. The gasket or sleeve 128g can be cylindrical and may have a flange that can be compressed by the block clamp 29 that the wires 128 pass through. FIG. 5E illustrates the closed/filled portal 127 with the cable 26 that merges into wires 128.

Although shown for one footswitch 25 in FIGS. 5B-5D, typically each has the same splash-resistant configuration, e.g., perimeter seal 125 and portal 127 with a solid filler 130, e.g., using a sleeve or gasket 128g and a filler/sealant 130.

The footswitch 25 is typically used outside of the sterile field in a surgical MRI, so it is not required to withstand sterilization processes (unlike the driver handpiece 10 and connector cable 15).

The connector cable or cord 15 transfers electrical signals and power from the control unit 20 to the motor M inside the driver handpiece 10. The cable 15 can have a plurality of conductors, typically between about 7-10 conductors (depending on whether a trigger and/or footswitch configuration is provided as a user control). The cable 15 can have a connector 15c with pins on each end, and typically has at least a 10 foot length. This provides adequate length to run from the handpiece driver 10 to the driver control unit 20. The control unit 20 can be placed outside the gauss line G-G (FIGS. 7A, 7B), and is also typically placed outside the sterile field and away from the scanner magnet 300 (FIGS. 7A, 7B).

The cable 15 can be configured to have identical connectors 15c on both ends for avoiding confusion as to which end plugs into the handpiece 10 versus the control unit. The cable 15 can be light weight and flexible so it does not add too much weight to the handpiece driver 10. The cable 15 can have between a 5-15 foot length. In some particular embodiment, the cable 15 can have a 10 foot length. The cable 15 can have a weight that is between about 0.1 pound and about 1 pound, typically about 0.2 pounds. Also, this allows it to be easily routed, typically on or above the floor, to the control unit 20. The cable 15 typically crosses the sterile/non-sterile barrier in the surgical suite. The cable 15 is configured to withstand repeated sterilizations and/or can be single-use disposable.

Summary of Exemplary Material Selection for System Components

| Component | Current Configuration | Alternatives |
|---|---|---|
| Driver Handpiece | | |
| Housing | PEEK | Ultem (PEI), ABS, Aluminum |
| | | (anodized), 316L SST |
| Drive Shaft | PEEK | Titanium, 316L SST, Aluminum |
| | | (anodized) |
| Chuck | PEEK | Titanium, 316L SST, Aluminum |
| | | (anodized) |
| Gears | Glass-filled Acetal | Brass, Aluminum |
| Speed Control Knob | Aluminum | PEEK, Ultem, ABS |
| Cable Receptacle | Nylon 6/6 Housing, Brass Pins | Brass, PEEK, PEI, Aluminum |
| Motor | Various | N/A |
| Control Box | | |
| Enclosure | ABS | N/A |
| Control Board | Various | N/A |
| Cable Receptacles | Nylon - Outer/Pin Housing | Outer Housing - PEEK, PEI, |
| | Pins - Tin-plated Brass | Polysulfone |
| | | Pin Housing - ABS, PEI |
| | | Pins - N/A |
| Foot Switch | | |
| Enclosure | ABS | Aluminum (anodized), 316 SST |
| Mounting Plate | Aluminum | 316 SST |
| Cable | Jacket - Polyurethane, PET | Jacket - PVC, Silicone |
| | Wire - Copper, PTFE | Wire - N/A |
| Cable Plug | Nylon - Outer/Pin Housing | Outer Housing - PEEK, PEI, |
| | Pins - Tin-plated Brass | Polysulfone, Aluminum |
| | | Pin Housing - ABS, PEI |
| | | Pins - N/A |
| Connector Cable | | |
| Cable | Jacket - Polyurethane, PET | Jacket - PVC, Silicone |
| | Wire - Copper, PTFE | Wire - N/A |
| Plugs | Nylon 6/6 Outer and Pin | Outer Housing - PEEK, PEI, |
| | Housing | Polysulfone, Aluminum |
| | Pins - Tin-plated Brass | Pin Housing - ABS, PEI |
| | | Pins - N/A |

The alternatives noted as N/A means that while alternative materials are available, they are not currently being considered for production.

Exemplary components and use of the driver 10 and methods according to embodiments of the present invention will be further described. The head includes an outer skin (and other soft tissue) layer (referred to herein as the scalp), a skull, and underlying brain tissue. The skull includes an outer compact bone layer, an inner compact bone layer, and a spongy bone layer between the compact bone layers. According to some embodiments, the drilling can occur with the patient's head in or adjacent a bore of a high-field magnet of an MRI scanner as shown in FIG. 7A, 7 (can be open bore or closed bore magnets). The patient “P” can be on an MR Scanner gantry or bed 300b.

Typically, as shown in FIGS. 7A and 7B, the patient's head is in a head fixation assembly 150 that may include head coils and typically includes one or more head restraints including a belt and bone screws to inhibit patient movement during surgery. See, e.g., U.S. patent application Ser. No. 12/685,849, the contents of which are hereby incorporated by reference as if recited in full herein. The head fixation assembly 150 can have any suitable configuration and the embodiment shown is by way of example only.

FIG. 7A illustrates that the drilling may occur while the patient is fully retracted in the bore 310 of the magnet of the MR Scanner 300. FIG. 7B illustrates that the gantry may be extended a distance outside the bore during use of the driver 10.

In some embodiments, a patient can be placed in an MRI scanner room. The patient can be placed on a gantry for retraction into the Scanner bore. A hand-held powered driver is placed in contact with a target location of the patient. A user triggers a power input (e.g., using a foot pedal or hand trigger), causing a drill bit of the driver to enter bone of the patient or driving a screw into bone of the patient.

Optionally, a head fixation frame can be attached to the head of the patient before placing the driver. FIGS. 8 and 9 illustrate the driver 10 used to screw a bone screw into bone (e.g., skull). The bone screws can hold a base 500b of a trajectory guide frame 500.

In some embodiments, the driver 10 and methods form a part of or operate with MRI compatible interventional systems. The driver can be provided with a set of interventional tools for a particular procedure type. In some embodiments, the MRI compatible interventional systems include the driver 10 with trajectory guide systems and/or apparatus and related components and methods. According to some embodiments, the trajectory guide apparatus and methods are frameless stereotactic trajectory guide apparatus that may be particularly suitable for deep brain interventional procedures, but may be used in other target anatomical locations as well.

Some embodiments of the invention are directed to MRI interventional procedures and provide interventional tools and/or therapies that may be used to locally place surgical interventional objects, tools or therapies in vivo to site specific regions using an MRI system. The interventional tools can be used to define an MRI-guided trajectory or access path to an in vivo treatment site.

In some embodiments, MRI can be used to visualize (and/or locate) a therapeutic region of interest inside the brain and utilize an MRI to visualize (and/or locate) an interventional tool or tools that will be used to deliver therapy and/or to place a permanently implanted device that will deliver therapy. Then, using the imaging data produced by the MRI system regarding the location of the therapeutic region of interest and the location of the interventional tool, the system and/or physician can make positional adjustments to the interventional tool so as to align the trajectory of the interventional tool, so that when inserted into the body, the trajectory of the interventional tool will intersect with the therapeutic region of interest. With interventional tool now aligned with the therapeutic region of interest, an interventional probe can be advanced, such as through an open lumen inside of the interventional tool, so that the interventional probe follows the trajectory of the interventional tool and proceeds to the therapeutic region of interest. The interventional tool and the interventional probe may or may not be part of the same component or structure.

Tools, methods and systems in accordance with the present invention may be used with apparatus and methods as described in one or more of the following patent applications: U.S. Provisional Patent Application No. 60/933,641, filed Jun. 7, 2007; U.S. Provisional Patent Application No. 60/974,821, filed Sep. 24, 2007; and PCT Application No. PCT/US2006/045752, published as PCT Publication No. WO/2007064739 A2, and U.S. patent application Ser. No. 12/134,412, filed Jun. 6, 2008, the disclosures of which are hereby incorporated by reference.

According to some embodiments, instrumentation and equipment are inserted through a targeting cannula to execute a diagnostic and/or surgical procedure. According to some embodiments, the procedure includes a deep brain stimulation procedure wherein one or more electrical leads are implanted in a patient's brain. The apparatus described herein can serve to designate an entry point into a patient for an established trajectory for installing the lead or leads or other interventional devices such as, for example, but not limited to, ablation probes, injection catheters and the like.

Some embodiments can be configured to deliver tools or therapies that stimulate a desired region of the sympathetic nerve chain. Other uses inside or outside the brain include stem cell placement, gene therapy or drug delivery for treating conditions, diseases, disorders or the like. Some embodiments can be used to treat tumors.

Some embodiments can be used with systems to deliver bions, stem cells or other target cells to site-specific regions in the body, such as neurological target and the like. In some embodiments, the systems deliver stem cells and/or other cardio-rebuilding cells or products into cardiac tissue, such as a heart wall via a minimally invasive MRI guided procedure, while the heart is beating (i.e., not requiring a non-beating heart with the patient on a heart-lung machine). Examples of known stimulation treatments and/or target body regions are described in U.S. Pat. Nos. 6,708,064; 6,438,423; 6,356,786; 6,526,318; 6,405,079; 6,167,311; 6,539,263; 6,609,030 and 6,050,992, the contents of which are hereby incorporated by reference as if recited in full herein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention. Therefore, it is to be understood that the foregoing is illustrative of the present invention and is not to be construed as limited to the specific embodiments disclosed, and that modifications to the disclosed embodiments, as well as other embodiments, are intended to be included within the scope of the invention.

That which is claimed is:

1. A surgical motor-powered driver, comprising:
a hand-held driver handpiece comprising a non-ferromagnetic housing, wherein the driver housing is sterile for surgical use;
a non-magnetic motor in the housing;
a shaft in communication with the motor; and
a chuck extending from the housing adapted to serially, releasably hold a drill bit and a screw driver so that the drill bit or screw driver extends out from the chuck and is rotated by the motor.

2. The driver of claim 1, further comprising a control unit connected to the handpiece by a cable having a length sufficient to allow the control unit to be positioned remote from the handpiece outside a gauss line of an MRI suite while the handpiece is held in a magnetic field of a magnet of an MRI scanner.

3. The driver of claim 1, wherein the handpiece operates the shaft with between about 20-150 rpm and generates a maximum torque of about 6.2 in-lb.

4. The driver of claim 1, further comprising a speed increase gear train in the handpiece in communication with the motor, wherein the gear train has a gear ratio of 2:1 and can generate an output speed between about 40-300 rpm with a maximum torque of about 3.1 in-lb.

5. The driver of claim 1, wherein the handpiece has a barrel attached to a position-adjustable handle, wherein the handle can rotate from lockable orientations between about 0-90 degrees to be respectively in-line with the barrel or substantially orthogonal with the barrel.

6. The driver of claim 1, wherein the driver has a dual operating mode, including a drill mode and a screw driver mode, wherein the screw driver mode has a lower speed than the drill mode.

7. The driver of claim 6, further comprising a user interface control on the driver handpiece configured to allow a user to switch between drill and screw driver modes.

8. The driver of claim 2, further comprising at least one footswitch with a respective pedal attached to the control unit with a cable.

9. The driver of claim 1, wherein the handpiece comprises a trigger to direct the driver to operate.

10. The driver of claim 1, wherein the handpiece is reusable in sterile medical environments and is configured to withstand a plurality of autoclaving and/or Ethylene Oxide (“EtO”) sterilization processes so as to remain functional and not deteriorate.

11. The driver of claim 1, wherein the footswitch has a non-ferromagnetic enclosure with cooperating housing members that can move relative to each other to allow the pedal to operate, and wherein an O-ring and/or gasket resides between the cooperating housing members allowing the relative movement to inhibit liquid entry into the enclosure to thereby provide a splash-resistant configuration.

12. The driver of claim 1, further comprising a cable attached to the handpiece to connect the motor to a control unit, wherein the cable and handpiece are configured to be reusable and withstand a plurality of sterilizations.

13. The driver of claim 1, wherein the handpiece has a barrel and a handle, wherein the handle is pivotably attached to the barrel at a pivot on a rear end portion of the barrel, and wherein the handle has a channel that slidably travels over a curved outer surface of the barrel a distance away from the pivot.

14. The driver of claim 13, wherein the curved outer surface includes a fin that projects radially outward a distance beyond an adjacent curved surface of the rear end portion of the barrel.

15. The driver of claim 13, further comprising an indexing control mechanism configured to allow a user to controllably adjust an orientation of the handle relative to the barrel over a plurality of positions.

16. The driver of claim 15, wherein the fin has a circumferentially extending slot that receives a pin of the indexing control mechanism.

17. The driver of claim 1, further comprising a control unit remote from the handpiece and a connector cable attached to and connecting the handpiece and the control unit, wherein the control unit has a controller that controls operation of the motor including forward/reverse operation, speed control, start/stop and system power.

18. The driver of claim 17, wherein the control unit has a housing of non-ferromagnetic material with connections for main power and the connector cable.

19. A method of inserting a bone screw or forming an aperture in bone of a patient during an MRI guided surgical procedure, comprising:
(a) placing a patient on a patient support surface in an MR Scanner room;
(b) holding a motor-driven driver against a target location of a patient while the patient is in the MR Scanner room;
(c) electrically powering the driver; and
(d) drilling an aperture in target bone of the patient or driving a bone screw into bone of a patient in response to the electrically powering step.

20. The method of claim 19, further comprising allowing a user to depress at least one pedal of a footswitch in communication with the driver to cause the driver to carryout step (d).
